(19) United States
(12) Patent Application Publication
(10) Pub. No.: US 2022/0142491 A1
(43) Pub. Date: May 12, 2022

(54) COMPUTER-ASSISTED METHOD AND SYSTEM FOR REMOTE MONITORING OF PATIENTS

(71) Applicant: Exal Bone Sp. z o.o., Jasionow (PL)

(72) Inventors: Mateusz Siwak, Lodz (PL); Lukasz Piotrowski, Lodz (PL)

(73) Assignee: Exal Bone Sp. z o.o., Jasionow (PL)

(21) Appl. No.: 17/139,629

(22) Filed: Dec. 31, 2020

(30) Foreign Application Priority Data
Nov. 9, 2020 (PL) 435926

(51) Int. Cl. A61B 5/0205; A61B 5/00; A61B 5/145

(52) U.S. Cl. CPC A61B 5/02055; A61B 5/746; A61B 5/7275; A61B 5/0004; A61B 5/14542

(57) ABSTRACT

A computer-assisted method is for remote monitoring of patients. The patient's health condition is monitored by analyzing results of measurement of health parameters entered in a computer system manually or through automated measuring sensors. Once the specific conditions are satisfied, a medical hazard alert is generated. Before monitoring is started, the data on a patient's health condition is downloaded and the patient is allocated to a specific risk group. A notification on the necessity to perform measurement of health parameters is generated with the frequency dependent on the risk group assigned to the patient. The measured health parameters are analyzed taking into account the risk group to which the patient was allocated.

The subject of the invention is a method and system for remote monitoring of patients.

Mass illnesses triggered by contagious diseases may require streamlined systems for remote monitoring of patients to reduce the workload on healthcare facilities.

For example, such necessity emerged recently due to the COVID-19 pandemic, where in many countries the healthcare facilities were overwhelmed with work, and in some countries the number of patients trying to get to the healthcare facilities exceeded the available capacity.

For some patients (in case of for COVID-19, for majority of patients), the course of infection is mild or moderate and does not require hospitalization. Unfortunately, subjective feelings of shortness of breath in conjunction with fever resulted in massive registrations of patients who did not need hospitalization. Only a small number of patients develop a severe form of disease, which requires hospitalization, and in a smaller group of patients it evolves into a critical condition. In the early stage of infection, it is difficult to determine whether a patient will need hospitalization. However, it is essential to diagnose patient whose condition deteriorated (often unnoticed) to such requiring hospitalisation so that medical care can be provided as early as possible to eliminate development of the critical condition.

It is therefore necessary to develop a system to remotely monitor and triage patients in order to efficiently identify those who are most likely to need treatment at a healthcare facility.

The subject of the invention is a computer-assisted method for remote monitoring of patients, in which the patient's health condition is monitored in a computer system by analysing measurements of health parameters entered in the system manually or through automated measuring sensors, and in the event of specific conditions being detected a medical hazard alert is generated, wherein the method is characterized in that:
before monitoring is started the data on patient's health condition is downloaded and accordingly the patient is assigned to a specific risk group
a notification of the necessity to perform measurement of health parameters is generated with the frequency dependent on the risk group to which the patient was assigned
the measured health parameters are analysed taking into account risk group to which the patient was assigned.

Preferably, health hazard boundary alert is generated in case the value of a health condition parameter exceeds the critical value.

Preferably, the boundary value of at least one health condition parameter is dependent on the measured value of at least one other health condition parameter.

Preferably, a combined health hazard alert is generated if a specific change in the measured values of at least two health condition parameters is detected relative to earlier measurements.

Preferably, in order to monitor patients for whom SARS-CoV-2 infection is suspected, a risk factor is assigned to the monitored patient, the value of which is determined by the following symptoms:
nicotinism: +2 points
Body Mass Index (BMI):
   >25: +1 point
   >30: +2 points
   >35: +3 points
   >40: +4 points
Age:
   >50: +1 point
   >60: +2 points
   >70: +3 points
Occurrence of at least one cardiovascular disease : +2 points
Occurrence of noninsulin-dependent diabetes mellitus (NIDDM): +1 point
Occurrence of insulin-dependent diabetes mellitus and >40 years of age: +2 points 3/23
Occurrence of at least one respiratory disease: +1 point
Occurrence of active cancer: +1 point
Immunity disorders: +1 point

Then based on the calculated risk factor, the patient is assigned to one of the following risk groups:
No risk group: 0 points
Low risk group: 1-2 points
Medium risk group: 3-5 points
High risk group: over 5 points
High pulmonary risk group: over 3 points for patients suffering from pulmonary diseases and having initial oxygen saturation equal or less than 95%.

Preferably, for patients from no risk group:
The measurement is taken every 6 hours
Boundary alert is generated if: the oxygen saturation falls below 95% or below 94% if there is a cough, a respiratory rate increases above 30 breaths per minute or heart rate increases above 120 beats per minute, while where the patient takes beta blockers the heart rate alert value is decreased by 20 and if the patient has got a temperature above 37° C., the alert value for the heart rate is increased by 8 per each degree above said temperature.
A combined alert is generated if the following occur simultaneously: the 4% decrease in the oxygen saturation in a single or several consecutive measurements, with no oxygen saturation increase in subsequent measurements, respiratory rate increases by 10 relative to the average and heart rate increases by 50% relative to the average.

Preferably, for patients from low risk group:
The measurement is taken every 5 hours
Boundary alert is generated if the oxygen saturation falls below 94% or below 93% if there is a cough, a respiratory rate increases above 30 breaths per minute or heart rate increases above 120 beats per minute, while where the patient takes beta blockers the heart rate alert value is decreased by 20 and if the patient has got a temperature above 37° C., the alert value for the heart rate is increased by 8 per each degree above said temperature.
A combined alert is generated if the following occur simultaneously: the 4% decrease in the oxygen saturation in a single or several consecutive measurements, with no oxygen saturation increase in subsequent measurements, respiratory rate increases by 10 relative to the average and heart rate increases by 50% relative to the average.

Preferably, for patients from medium risk group:
The measurement is taken every 4 hours
Boundary alert is generated if the oxygen saturation falls below 92% or below 91% if there is a cough, a respiratory rate increases above 30 breaths per minute or heart rate increases above 130 beats per minute, while where the patient takes beta blockers the heart rate alert value is decreased by 15 and if the patient has got a temperature above 37° C., the alert value for the heart rate is increased by 8 for each degree above said temperature.
A combined alert is generated if the following occur simultaneously: the 3% decrease in the oxygen saturation in a single or several consecutive measurements, with no oxygen saturation increase in subsequent measurements, respiratory rate increases by 10 relative to the average and heart rate increases by 40% relative to the average.

Preferably, for patients from high risk group:
The measurement is taken every 2 hours
Boundary alert is generated if the oxygen saturation falls below 92% or below 90% if there is a cough, a respiratory rate increases above 30 breaths per minute or heart rate increases above 130 beats per minute, while where the patient takes beta blockers the heart rate alert value is decreased by 15 and if the patient has got a temperature above 37° C., the alert value for the heart rate is increased by 8 for each degree above said temperature.
A combined alert is generated if the following occur simultaneously: the 3% decrease in the oxygen saturation in a single or several consecutive measurements, with no oxygen saturation increase in subsequent measurements, respiratory rate increases by 12 relative to the average and heart rate increases by 40% relative to the average.

Preferably, for patients from high pulmonary risk group:
The measurement is taken every 4 hours
Boundary alert is generated if the oxygen saturation falls below 89% or below 87% if there is a cough, a respiratory rate increases above 40 breaths per minute or heart rate increases above 130 beats per minute, while where the patient takes beta blockers the heart rate alert value is decreased by 15, and if the patient has got a temperature above 37° C., the alert value for the heart rate is increased by 8 for each degree above said temperature.
A combined alert is generated if the following occur simultaneously: the 5% decrease in the oxygen saturation in a single or several consecutive measurements, with no oxygen saturation increase in subsequent measurements, respiratory rate increases by 12 relative to the average and heart rate increases by 40% relative to the average.

Furthermore, the subject of the invention is a computer-assisted system for remote patients' monitoring, which system includes the patient database connected to analytic and communication servers which use a telecommunication infrastructure to communicate with patients' and medical staff's devices, wherein said system is characterised in that:
Patient database includes a table of patients with the data about the patients' health condition and the risk group to which the patient was assigned;
Communication server is adapted to generate notifications of the necessity to perform measurement of health parameters with the frequency dependent on the risk group to which the patient was assigned;
Analytic server is used to analyse the measured health parameters taking into account risk group to which the patient was assigned.

Preferably, the system is adapted to implement the method described above.

According to the invention, the system enables early identification of patients whose health parameters measured indicate the risk of decompensation and development of severe course of COVID 19 infection, potentially resulting in a critical condition. Such patient data can be efficiently passed to medical staff, who can then undertake medical actions appropriate for the given patient, for example a referral to hospital. This optimises health care resources, with the possibility to give up hospitalisation and put a patient on observation, keeping hospital beds only for the patients who need them most.

The Invention has been described in embodiment using representation in figures, in which:

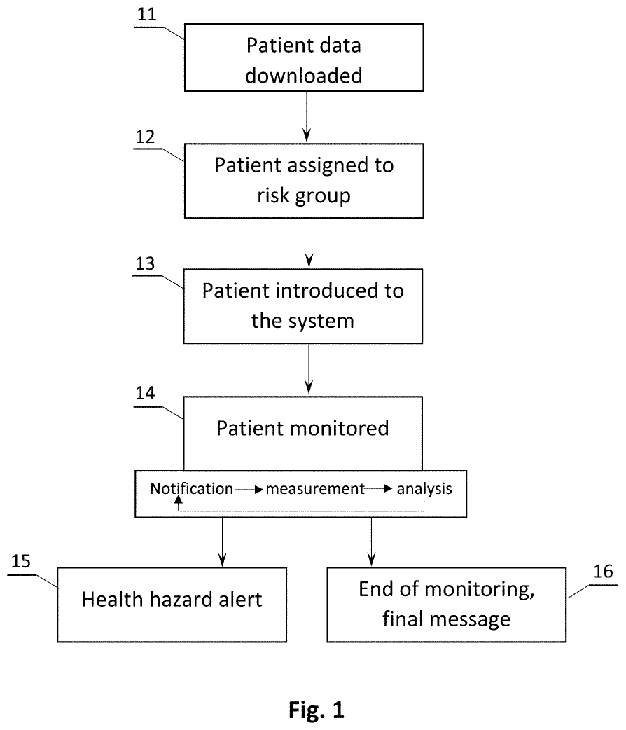

FIG. 1 shows the scheme of the patient monitoring method

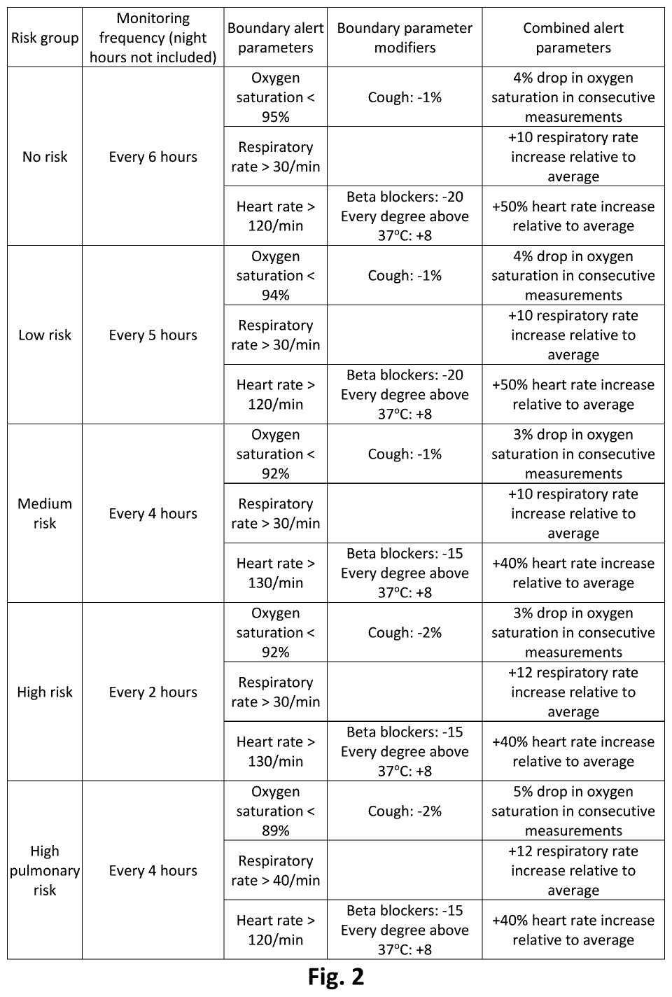

FIG. 2 shows factors leading to alert generation

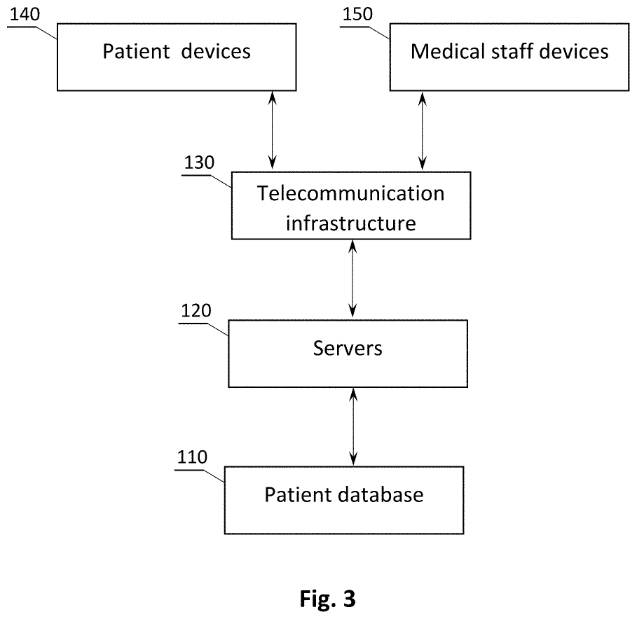

FIG. 3 shows patient monitoring system

FIG. 4 shows an example of database structure

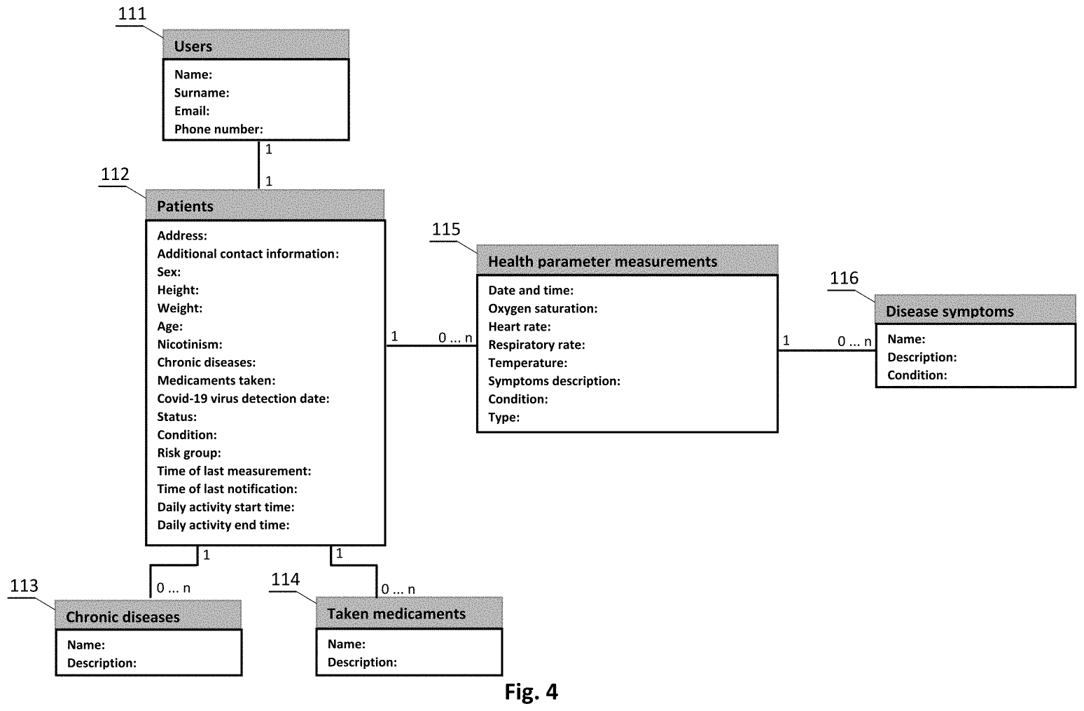

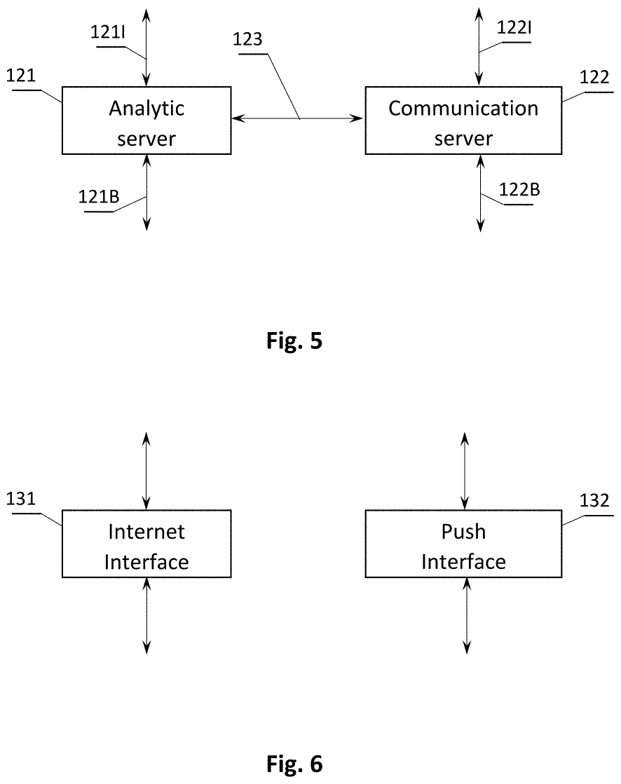

FIG. 5 shows examples of system servers

FIG. 6 shows examples of communication system interfaces

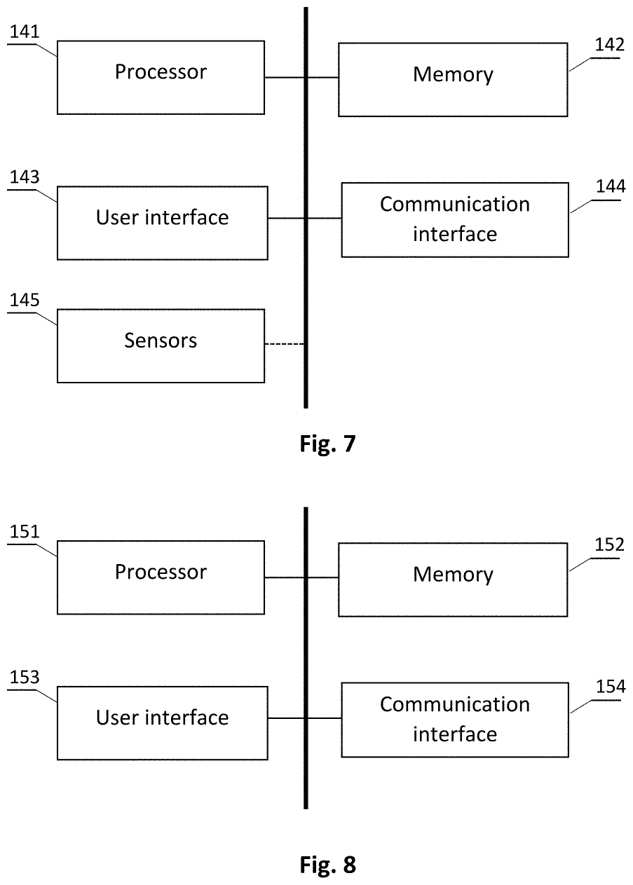

FIG. 7 shows example patient device used in the system

FIG. 8 shows example medical staff's device used in the system

The embodiment presented here will be described based on exemplary monitoring of patients who are suspected of SARS-CoV-2 infection. However, with appropriate modifications, the system can also be used to monitor and assess patients with regard to other mass diseases.

Fundamentally, the system consists in monitoring the patients and detection of symptoms which qualify the patient for more extensive medical assistance, for example the need for hospitalization. The stages of the method are shown in FIG. 1.

In the first stage 11 patient data including personal data and health condition information is downloaded. The data can be downloaded by the medical staff using medical staff device 150 during a medical appointment. Alternatively, this data can be manually entered into the system by the patient using an appropriate application or webpage interface with the help of patient device 140, for example after the patient is diagnosed with a virus infection. In particular, the data necessary to assign the patient to a specific risk group is downloaded, as described below.

Subsequently in stage 12, as based on the health condition information, the patient is assigned to a risk group.

For example, by monitoring patients with regards to SARS-CoV-2 virus infection, one can determine risk factor for the patient, by assigning them a value associated with the following symptoms:
nicotinism: +2 points
Body Mass Index (BMI):
   >25: +1 point
   >30: +2 points
   >35: +3 points
   >40: +4 points
Age:
   >50: +1 point
   >60: +2 points
   >70: +3 points
Occurrence of at least one cardiovascular disease (such as coronary disease, history of heart attack or stroke, heart failure): +2 points
Occurrence of noninsulin-dependent diabetes mellitus (NIDDM): +1 point
Occurrence of insulin-dependent diabetes mellitus and >40 years of age: +2 points
Occurrence of at least one respiratory disease (such as asthma, chronic obstructive pulmonary disease, cystic fibrosis): +1 point
Occurrence of active cancer: +1 point
Immunity disorders (for example patients after transplantations taking immunosuppressant drugs, patients with congenital immune deficiency): +1 point

For example, a 51-year old patient with BMI of 36, suffering from coronary disease, with heart attack history and suffering from insulin-dependent diabetes mellitus will have a risk factor of 1+3+2+2=8 points assigned placing the example patient in the high risk group.

Based on the calculated risk factor value, the patient is assigned to one of the following risk groups:
No risk group: 0 points
Low risk group: 1-2 points
Medium risk group: 3-5 points
High risk group: >5 points
High pulmonary risk group: >3 points for patients suffering from pulmonary diseases and have initial oxygen saturation equal or less than 95%.

Subsequently, in stage 13, the patient is entered into the system. For patients from the no risk group the medical staff can decide not to ienter the patient into the system so as to conserve system resources.

In stage 14, patient health condition parameters are monitored. The monitoring is performed with the help of appropriate sensors e.g. thermometer to measure body temperature, pulse oximeter to measure oxygen saturation and stopwatch to measure respiratory rate. The measurements are taken with a specific frequency dependent on the risk group, to which the patient has been assigned. The system generates reminders to take the measurements, which can be delivered to the patient by an application run on the patient device 140 and/or through a communication server 122 as an SMS sent by Push type communication interface via GSM network to the patient device 140. The measurements can be taken by the patients themselves and entered into the system through a dedicated application run on the patient device 140. Some and/or all the measurements can be taken automatically if the patient device 140 is equipped with appropriate sensors or thereto connencted (e.g. wearables). The measurement results entered are analysed by the analytic server 121.

If, as a result of analysis, the analytic server 121 detects that specific conditions are satisfied, it generates a health hazard alert in stage 15 (through a signal or a message), which is sent to the medical staff device 150. This allows a doctor assigned to the patient to get in touch with the patient or, a medical dispatch to send an ambulance to transport the patient to the hospital.

If medical examination or teleconsultation by medical staff concludes that the alert was caused by an erroneously filled health condition survey which assigned the patient to the wrong risk group or occurred medical factors not accounted for in the survey but requiring a change of the risk level, such may be manually changed according to the decision of the operator. Subsequently, in case where the patient is hospitalised, the monitoring can be ended as the care over the patient is then taken over directly by an appropriate healthcare facility.

Finally, when the period of time during which the patient should be monitored ends (for example 3 days after disappearance of COVID-19 symptoms as decided by a family physician), the monitoring ends and a final message is sent to the patient informing that there is no need for further monitoring since the patient has either already recovered or the risk of occurrence of medical symptoms is negligible.

Analytic server 121 works based on the decision algorithm, in which two types of alerts can occur: boundary alerts indicating detection of specific values, or combined alerts indicating exceedance of the allowable parameter change value over predefined time period. The values taken by the algorithm for the patients in the specific risk groups have been shown in FIG. 2 and will be described below.

For patients from no risk group:
The measurement is taken every 6 hours
Boundary alert is generated if: the oxygen saturation falls below 95% or below 94% if there is a cough, a respiratory rate increases above 30 breaths per minute or heart rate increases above 120 beats per minute, while where the atient takes beta blockers the heart rate alert value is decreased by 20 and if the patient has got a temperature above 37° C., the alert value for the heart rate is increased by 8 for each degree above said temperature.
A combined alert is generated if the following occur simultaneously: the 4% decrease in the oxygen saturation in a single or several consecutive measurements, with no oxygen saturation increase in subsequent measurements, respiratory rate increases by 10 relative to the average and heart rate increases by 50% relative to the average.

The average is calculated for a specific number of earlier measurements, for example 5 or more measurements. If the number of measurements is lower, the conditions for the combined alert are not checked.

For patients from low risk group:
The measurement is taken every 5 hours
Boundary alert is generated if the oxygen saturation falls below 94% or 93% if there is a cough, a respiratory rate increases above 30 breaths per minute or heart rate increases above 120 beats per minute, while where the patient takes beta blockers the heart rate alert value is decreased by 20 and if the patient has got a temperature above 37° C., the alert value for the heart rate is increased by 8 for each degree above said temperature.
A combined alert is generated if the following occur simultaneously: the 4% decrease in the oxygen saturation in a single or several consecutive measurements, with no oxygen saturation increase in subsequent measurements, respiratory rate increases by 10 relative to the average and heart rate increases by 50% relative to the average.

Compared with the patients from no risk group, occurrence of lower oxygen saturation values is accepted in this group before the boundary alert is generated as it can result from the pre-existing risk factors in these patients.

For patients from medium risk group:
The measurement is taken every 4 hours
Boundary alert is generated if the oxygen saturation falls below 92% or 91% if there is a cough, a respiratory rate increases above 30 breaths per minute or heart rate increases above 130 beats per minute, while where the patient takes beta blockers the heart rate alert value is decreased by 15 and if the patient has got a temperature above 37° C., the alert value for the heart rate is increased by 8 for each degree above said temperature.
A combined alert is generated if the following occur simultaneously: the 3% decrease in the oxygen saturation in a single or several consecutive measurements, with no oxygen saturation increase in subsequent measurements, respiratory rate increases by 10 relative to the average and heart rate increases by 40% relative to the average.

Compared with the patients from low risk group, occurrence of lower oxygen saturation and higher heart rate values is accepted in this group before the boundary alert is generated as it can result from the pre-existing risk factors in these patients. In turn the alert is generated for lower, compared with low risk group, deviations from initial parameters.

For patients from high risk group:
The measurement is taken every 2 hours
Boundary alert is generated if the oxygen saturation falls below 92% or 90% if there is a cough, a respiratory rate increases above 30 breaths per minute or heart rate increases above 130 beats per minute, while where the patient takes beta blockers the heart rate alert value is decreased by 15 and if the patient has got a temperature above 37° C., the alert value for the heart rate is increased by 8 for each degree above said temperature.
A combined alert is generated if the following occur simultaneously: the 3% decrease in the oxygen saturation in a single or several consecutive measurements, with no oxygen saturation increase in subsequent measurements, respiratory rate increases by 12 relative to the average and heart rate increases by 40% relative to the average.

Compared with the patients from medium risk group, occurrence of lower oxygen saturation is accepted in this group before the boundary alert is generated if accompanied by a cough as it can result from the pre-existing risk factors for these patients. Larger deviations from average value for respiratory rates are also accepted.

For patients from high pulmonary risk group:
The measurement is taken every 4 hours
Boundary alert is generated if the oxygen saturation falls below 89% or 87% if there is a cough, a respiratory rate increases above 40 breaths per minute or heart rate increases above 130 beats per minute, while where the patient takes beta blockers the heart rate alert value is decreased by 15, and if the patient has got a temperature above 37° C., the alert value for the heart rate is increased by 8 for each degree above said temperature.
A combined alert is generated if the following occur simultaneously: the 5% decrease in the oxygen saturation in a single or several consecutive measurements, with no oxygen saturation increase in subsequent measurements, respiratory rate increases by 12 relative to the average and heart rate increases by 40% relative to the average.

Compared with the patients from high risk group, occurrence of lower oxygen saturation and higher respiratory rate is accepted in this group as for these patients, due to pulmonary diseases, there is initial low oxygen saturation and high respiratory rate. Larger deviations from average value for oxygen saturation and respiratory rates are also accepted.

To minimise the number of false alarms potentially triggered by measurement error or incorrect data input, in case the condition for boundary or combined alert is satisfied, a request to repeat the measurements can be sent to the patient, and then the emergency message is sent only when the verification measurement confirms the earlier measurement (i.e. the conditions for alert generation are correctly met) or when the patient does not take the verification measurement within a prescribed time (for example within half an hour).

In particular, the necessity to take extra measurement can be flagged in case ‘artifacts’ are detected, i.e. significant deviation of one parameter, not accompanied by change in any other parameter. For example, if so far a patient has had oxygen saturation between 97-99% which then rapidly dropped to 84%, but this sudden drop was not accompanied by an increase in the heart rate or respiratory rate, this might indicate occurrence of a measurement ‘artifact’ and therefore necessity to repeat the measurement.

The period during which measurements are taken can be confined to the patient's daily activity period, indicated in the patient data table 112. For example, a patient in medium risk group, whose daily activity starts at 8 am and finishes at 10 pm, may take the first measurement at 8 am, second at 12 am, third at 4 pm and fourth at 8 pm.

If, the measurement data is not entered into the system at the predicted time, the system (by means of communication server) can generate a reminder (via application or push type communication SMS) to take measurement. The reminders can be generated cyclically, for example dependent on the risk group, for instance for the low risk group they can be generated every hour and for the high risk group every 30 minutes. If, after generating a specific number of reminders, the measurement results are still not entered into the system, the patient's status can be changed to ‘no measurement’.

In particular, a following reminder generation schedule can be taken:
For patients from no risk group: no SMS, Push type message in the application after 3 hours then every 1 hour, unless any of the earlier measurements was classified as an alert, then the procedure same as for low risk group should be implemented;
For patients from low risk group: SMS after 1 hour, Push message in the application every 1 hour, Blue Alert after 24 hours, unless any of the earlier measurements was classified as an alert, then the procedure same as for medium risk group should be implemented;
For patients from medium risk group: SMS after 30 minutes, Push message in the application every 30 minutes, Blue Alert after 12 hours;
For patients from high risk group: SMS after 30 minutes, Push message in the application every 30 minutes, Blue Alert after 6 hours;
For patients from high pulmonary risk group: SMS after 30 minutes, Push message in the application every 15 minutes, Blue Alert after 6 hours.

‘Blue Alert’ means that the system dispatcher is notified of denoting the patient as not taking measurements according to the system recommendations, in order to, for example, contact the patient.

An embodiment of computer-assisted system for remote monitoring of patients according to the invention is shown in FIG. 3.

The system comprises patient database 110, which can be arranged as a centralised or a distributed database. Patient database 110 can be arranged as relational database or any other appropriate type of database. An example of the structure of a database, arranged as relational database, is shown schematically in FIG. 4.

In the user table 111, basic information on system user is stored, which enables basic contact with the patient, such as name, surname, email, and phone number.

Patient database 112 contains more sensitive data about the patient, such as address, additional contact information, gender, weight, age, smoking habits, description of chronic diseases, description of medications taken by the patient, time of COVID-19 diagnosis, patient status, risk category assigned to the patient, time of the latest health condition measurement, time of latest notification about the need to take measurements, time when patient's daily activity starts and ends.

The patient status can take following values:
Monitored—the patient is under the care of the system
Observed—the patient is monitored and displayed as priority on the monitored patients list
Muted—the patient is normally monitored by the system but their measurements do not generate alerts for the medical staff, they do not appear on the list of patients at risk or patients without measurements
No measurements—the patient did not take several of the planned health condition measurements
At risk—the patient has been selected by the system for hospitalisation, awaiting reaction from medical staff
Selected for hospitalisation—the patient is still monitored but emergency medical service has been called to physically examine the patient and make a decision about hospitalisation
Archived—the patient is no longer monitored by the system, they do not appear on the patient list in the given region but can be searched for in the system. The system can differentiate archived patients as:
   Hospitalised—the patient undergoes treatment in the hospital—no further data collected/recorded
   Deceased—the medical staff marked the patient as deceased
   Recovered—the medical staff marked the patient as recovered
   Turned off—the monitoring of the patient was turned off by the medical staff
   Resigned—the patient resigned from monitoring on their own accord

The patient's health condition can take following values:
Normal—all health parameters are normal
At risk—the patient's health condition is alarming and medical help is likely to be necessary.

For each patient with a chronic disease, table 113 stores data on one or more diseases, including name and description of the diseases.

In addition, for each patient under medication table 114 stores data on one or more medications being taken, including name and description of the dosage.

For each patient with a health condition table 115 stores data on measurements, including day and time of measurement, oxygen saturation, respiratory rate, heart rate, body temperature, description of symptoms of the disease and type of measurement (for example planned measurement, requested measurement, spontaneous measurement).

For each measurement, table 116 stores data on the disease symptoms details, including symptom name, detailed symptom description and current state (for example: normal, no measurements, alert).

Apart from the aforementioned patient database 110 the system can contain number of other databases supplementary to the system operation, for example medical staff database (containing data on support users for the system operation), telecommunication operators database (containing data on communication channels) and other technical supplementary databases.

Servers 120 carrying out the main functionality of the system have access to the patient database 110. The servers can be centralised or distributed. Examples of server types have been shown in FIG. 5.

Analytic server 121 is adapted to analyse data saved in the patient database 110. It has communication channels 121B to communicate with patient database and communication channels 121I to communicate with telecommunication infrastructure 130. Analytic server is responsible for the procedure of data analysis and alert generation in stage 14.

Communication server 122 is adapted to enter data to the patient database 110 and reading data from this database 110. It has communication channels 122B to communicate with patient database and communication channels 121I to communicate with telecommunication infrastructure 130. The communication server is predominantly responsible for realisation of stage 14 in the procedure with regards to generating notifications (if sent through push message) and sending alerts to patients and medical staff if analytic server 121 generates an alert following of data analysis. Communication server can also enable patients and medical staff to access the data saved in the database. For example, the patient can browse history of their measurements. On the other hand, the medical staff can browse patients (based on criteria such as name, status, measurement results), patient results or measurements of patient groups, etc.

Furthermore, communication channels 123 can be placed to enable data exchange between servers 121 and 122.

Communication interfaces 130 are used to send data between system users, in particular between patients and medical staff and servers 120. Examples of communication servers 130 have been shown in FIG. 6.

Internet interface 131 is adapted to enable communications via internet browser, for example to enable the users to enter measurement results to the database 110 or to read data from the database 110, as well as to enable the medical staff to access patient data. Alternatively or additionally, analogical interfaces to enable communication through other data exchange networks can be used.

Push interface 132 is adapted to send reminder messages and alerts to patients or medical staff through communication channels such as Internet or mobile phone network (for example SMS gateway for GSM network), for example messages reminding to take measurements or alerts on bad health condition of the patient.

A diagram of an exemplary patient device 140 for daily system operation is shown on FIG. 7. Patient device 140 is equipped with typical systems, such as processor to run programs saved in the memory 142, user interface 143 to communicate with the user and communication interface 144 to send the data. Device 140 can be a general purpose device, for example a personal computer (PC) or a smartphone and might not be equipped with sensors 145 to measure health condition—in such case the patient should use separate sensors (for example their own thermometer, pulse oximeter and stopwatch to measure respiratory rate) and enter the measurement results to the device 140 manually. Alternatively, patient device 140 can be equipped with sensors 145 and then the results of these measurements can be registered automatically. The results of the measurements are entered into the system through appropriate application run on the patient's device 140, which sends the results through communication infrastructure 130 to servers 120, which save them in the database 110. Furthermore, patient device 140 can receive, through telecommunication infrastructure 130 push messages generated by the servers 120, for example informing about the need to take measurements, which can be presented to the user through user interface 143 in the form of graphical and/or sound message.

A diagram of an exemplary medical staff device 150 for system operation is shown in FIG. 8. Medical staff device 150 is equipped with typical systems such as processor to run programs saved in memory 152, user interface 152 to communicate with the user and communication interface 154 to send the data. Device 150 can be a general purpose device, for example a personal computer (PC) or a smartphone. Medical staff communicates with the system and the servers through communication infrastructure 130 in order to read data from the database 110, with the help of dedicated application or website supported by a web browser. In addition, medical staff device 150 can receive, through communication infrastructure 130, push messages generated by servers 120, for example informing about the need to see the patient.

1. A computer-assisted method of remote monitoring of patients, the method comprising:
monitoring a patient's health condition in a computer system by analysing results of measurement of health parameters entered into the system either manually or through automated measuring sensors, wherein in the event of specific conditions being met, generating a medical hazard alert;
before monitoring is started, downloading data on patient's health condition and based on the data, allocating the patient to a specific risk group;
generating a notification on the necessity to perform measurement of health parameters with frequency dependent on a risk group assigned to the patient; and
analyzing the measured health parameters taking into account the risk group to which the patient was allocated.

2. The method according to claim 1 wherein a health hazard boundary alert is generated in case where a value of a recorded health condition parameter exceeds a boundary value.

3. The method according to claim 2 wherein the boundary value of at least one health condition parameter is dependent on the measured value of at least one other health condition parameter.

4. The method according to claim 1, wherein a combined health hazard alert is generated if a specific change in the measured values of at least two health condition parameters relative to earlier measurements is detected.

5. The method according to claim 1, wherein in order to monitor patients for whom SARS-CoV-2 infection is suspected, a risk factor is assigned to the monitored patient, the value of which is associated with the following symptoms:
nicotinism: +2 points
Body Mass Index (BMI):
>25: +1 point
>30: +2 points
>35: +3 points
>40: +4 points
Age:
>50: +1 point
>60: +2 points
>70: +3 points
Occurrence of at least one cardiovascular disease: +2 points
Occurrence of noninsulin-dependent diabetes mellitus (NIDDM): +1 point
Occurrence of insulin-dependent diabetes mellitus and >40 years of age: +2 points
Occurrence of at least one respiratory disease: +1 point
Occurrence of active cancer: +1 point
Immunity disorders: +1 point
after which, based on the calculated risk factor, the patient is assigned to one of the following risk groups:
No risk group: 0 points
Low risk group: 1-2 points
Medium risk group: 3-5 points
High risk group: over 5 points
High pulmonary risk group: over 3 points for patients suffering from pulmonary diseases and having initial oxygen saturation equal or less than 95%.

6. The method according to claim 5 wherein for patients from no risk group:
a measurement is taken every 6 hours;
a boundary alert is generated if the oxygen saturation falls below 95% or 94% if there is a cough, a respiratory rate increases above 30 breaths per minute or heart rate increases above 120 beats per minute, while where the patient takes beta blockers the heart rate alert value is decreased by 20 and if the patient has a temperature above 37° C., the alert value for the heart rate is increased by 8 for each degree above said temperature;
a combined alert is generated if the following occur simultaneously: a 4% decrease in the oxygen saturation in a single or several consecutive measurements, with no oxygen saturation increase in subsequent measurements, respiratory rate increases by 10 relative to the average and heart rate increases by 50% relative to the average.

7. The method according to claim 5, wherein for patients from low risk group:
a measurement is taken every 5 hours;
a boundary alert is generated if the oxygen saturation falls below 94% or 93% if there is a cough, a respiratory rate increases above 30 breaths per minute or heart rate increases above 120 beats per minute, while where the patient takes beta blockers the heart rate alert value is decreased by 20 and if the patient has got a temperature above 37° C., the alert value for the heart rate is increased by 8 for each degree above said temperature;
a combined alert is generated if the following occur simultaneously: the a 4% decrease in the oxygen saturation in a single or several consecutive measurements, with no oxygen saturation increase in subsequent measurements, respiratory rate increases by 10 relative to the average and heart rate increases by 50% relative to the average.

8. The method according to claim 5, wherein for patients from medium risk group:
a measurement is taken every 4 hours;
a boundary alert is generated if the oxygen saturation falls below 92% or 91% if there is a cough, a respiratory rate increases above 30 breaths per minute or heart rate increases above 130 beats per minute, while where the patient takes beta blockers the heart rate alert value is decreased by 15 and if the patient has got a temperature above 37° C., the alert value for the heart rate is increased by 8 for each degree above said temperature;
combined alert is generated if the following occur simultaneously: a 3% decrease in the oxygen saturation in a single or several consecutive measurements, with no oxygen saturation increase in subsequent measurements, respiratory rate increases by 10 relative to the average and heart rate increases by 40% relative to the average.

9. The method according to claim 5, wherein for patients from high risk group:
a measurement is taken every 2 hours;
a boundary alert is generated if the oxygen saturation falls below 92% or 90% if there is a cough, a respiratory rate increases above 30 breaths per minute or heart rate increases above 130 beats per minute, while where the patient takes beta blockers the heart rate alert value is decreased by 15 and if the patient has got a temperature above 37° C., the alert value for the heart rate is increased by 8 for each degree above said temperature;
a combined alert is generated if the following occur simultaneously: a 3% decrease in the oxygen saturation in a single or several consecutive measurements, with no oxygen saturation increase in subsequent measurements, respiratory rate increases by 12 relative to the average and heart rate increases by 40% relative to the average.

10. The method according to claim 5, wherein for patient from high pulmonary risk group:
a measurement is taken every 4 hours;
a boundary alert is generated if the oxygen saturation falls below 89% or 87% if there is a cough, a respiratory rate increases above 40 breaths per minute or heart rate increases above 130 beats per minute, while where the patient takes beta blockers the heart rate alert value is decreased by 15, and if the patient has got a temperature above 37° C., the alert value for the heart rate is increased by 8 for each degree above said temperature;
combined alert is generated if the following occur simultaneously: a 5% decrease in the oxygen saturation in a single or several consecutive measurements, with no oxygen saturation increase in subsequent measurements, respiratory rate increases by 12 relative to the average and heart rate increases by 40% relative to the average.

11. A computer-assisted system for remote monitoring of patients said system comprising:
a patient database, to which analytic and communication servers are connected, with which, through telecommunication infrastructure devices of patients and medical staff communicate, wherein:
the patient database contains a patient table with the data on patient health condition and a risk group assigned;
a communication server is adapted to generate reminders to take measurements of patient health parameters with frequency dependent on the risk group to which the patient has been assigned;
an analytic server is adapted to analyse health parameter measurement results taking into account the risk group to which the patient has been assigned.

12. A computer-assisted system for remote monitoring of patients said system comprising:
a patient database, to which analytic and communication servers are connected, with which, through telecommunication infrastructure devices of patients and medical staff communicate, wherein:
the patient database contains a patient table with the data on patient health condition and a risk group assigned;
a communication server is adapted to generate reminders to take measurements of patient health parameters with frequency dependent on the risk group to which the patient has been assigned;
an analytic server is adapted to analyse health parameter measurement results taking into account the risk group to which the patient has been assigned;
wherein the system is adapted to carry out the method according to claim 1.
